(19) United States
(12) Patent Application Publication
(10) Pub. No.: US 2010/0198089 A1
(43) Pub. Date: Aug. 5, 2010

(54) METHODS AND SYSTEMS FOR PUMPING FLUIDS

(75) Inventors: Aharon Litovchick, Rehovot (IL); Ilya Litovchick, Rehovot (IL)

Correspondence Address: RECHES PATENTS, 211 North Union St., Suite 100, Alexandria, VA, 22314, US

(73) Assignee: CARD GUARD SCIENTIFIC SURVIVAL LTD, Rehovot (IL)

(21) Appl. No.: 12/526,953

(22) PCT Filed: Feb. 28, 2008
(86) PCT No.: PCT/IL2008/000259

§ 371 Date: Apr. 8, 2010

Related U.S. Application Data
(60) Provisional application No. 60/892,263, filed on Mar. 1, 2007.

(51) Int. Cl. A61B 5/0408

(52) U.S. Cl. 600/509

(57) ABSTRACT

An electrocardiographic system, the electrocardiographic system includes: (i) a processor, adapted to receive three electrocardiographic signals and to provide a cardiological problem indication in response to the three electrocardiographic signals, wherein each of the three electrocardiographic signals is detected between a different pair of electrodes out of a electrode group that consists of three electrodes located on a body of a patient; and (ii) an interface, adapted to transmit the cardiological problem indication received from the processor.

### RELATED APPLICATION

This application claims the priority of U.S. provisional patent Ser. No. 60/892,263, titled “Usage of adapted Nehb 3-lead bipolar leads for express ECG diagnostics”, Filed on Mar. 1, 2007.

### FIELD OF THE INVENTION

The invention relates to methods and systems for electrocardiography.

### BACKGROUND OF THE INVENTION

In 1938 W. Nehb proposed a 3-lead bipolar electrocardiography (ECG) technique with the leads defined as Nehb's A (anterior), Nehb's D (dorsalis) and Nehb's J (or Nehb's I—inferior). FIGS. 1a and 1b illustrate implementation of Nehb's prior art 3-lead bipolar ECG technique, wherein FIG. 1a illustrates a coronal view of a mail patient, and FIG. 1b illustrates a transverse cross section thereof. It is noted that recording in these leads could be accomplished by using any commercially available electrocardiograph.

According to Nehb's prior art electrocardiography, active electrodes are located on the chest in the following order: (a) first Nehb electrode 901 is placed in the second intercostal space to the right from sternum, corresponding to V1 for standard 12-lead ECG recording; (b) second Nehb electrode 902 is placed in the position corresponding to V4; and (c) third Nehb electrode 903 is placed in the position corresponding to V7.

According to said prior art 3-lead bipolar ECG technique, the ECG is recorded as if in the I standard lead would be defined as Nehb's D, which records the potential on the posterior left ventricle wall; II standard lead would produce Nehb's A which corresponds to the potential on the anterior wall of the left ventricle, and III standard lead would record Nehb's J, which reflects the potential on the diaphragmatic surface of heart.

Because the leads are placed on the chest in the immediate proximity to the heart aligned to the anatomic position of the heart, they are very sensitive and allow accurate diagnostics of different heart conditions including even those, which not always could be clearly detected by the standard 12-lead ECG system. For example especially characteristic diagnostic advantage for Nehb's leads is cases of myocardial ischemia in the left ventricle posterior wall.

Qualitative and quantitative parameters are taken into consideration when analyzing the ECG recorded by Nehb. Qualitative parameters are the same as in 12 leads ECG and conveniently include the size, the voltage and the shape of different waves P, Q-R-S, T, U; prolongation of different fragments like PR, QRS, ST or QT and their interrelationship like P-QRS association and dissociation, PR and ST deviations. In most cases QRS complex in Nehb's tracings of normal heart is recorded as qRS or rS, but occasionally it may be of qRs, qR, RS (Rs) morphology. In inferior lead (Nehb's J) Q wave is normally absent. Quantitative characteristics are the Q, R, S voltage, Q prolongation and Q/R and R/S ratio.

Table 1 illustrates quantitative characteristics of ECG parameters recorded by Nehb's technique.

TABLE 1

| | Lead (Nehb) | | | | | |
|---|---|---|---|---|---|---|
| | D | | A | | J | |
| Parameter | M | SD | M | SD | M | SD |
| Q Amplitude (mm) | 2.3 | 1.3 | 1.9 | 1.2 | — | — |
| Q Prolongation (sec) | 0.02 | 0.003 | 0.02 | 0.003 | — | — |
| R Amplitude (mm) | 14.7 | 4.5 | 21.0 | 6.2 | 6.1 | 2.9 |
| S Amplitude (mm) | 2.9 | 1.4 | 5.4 | 2.9 | 4.6 | 2.6 |
| Q/R Ratio | 0.16 | 0.1 | 0.09 | 0.05 | — | — |
| R/S Ratio | 6.3 | 3.6 | 5.4 | 5.4 | 2.1 | 2.7 |

It is noted that there is also data of the prior art also teaches of successful implementation of these leads for Holter-monitoring, as compared to ones typically used for this purpose (CM2, CMS, CR4, CS5 etc).

It is noted that Nehb's method is unjustly forgotten and deserves application at present, with required adaptations resulting from progress of the medical sciences. Most researchers that have used Nehb's leads agree that simplicity, high sensitivity, and information are great advantages of this ECG setup.

It is further noted that preliminary clinical data testifies to high reliability of the Nehb method when for urgent diagnostics, as correlated to the 12-lead electrocardiograph.

Additionally, current ambulatory electrocardiography devices (e.g. Holter monitors) implement no less than five electrodes (e.g. four signal electrodes and a ground electrode) for the detection of electrocardiographic information, and thus are cumbersome to attach to the body and to carry around, and the connecting of all the five electrodes takes time and could usually be carried out by a professional. It is clear to a person who is skilled in the art that there is a great need in reducing the number of electrodes in mobile electrocardiographic devices.

There is a need for reliable and simple means for electrocardiography, and especially for express-diagnostics of various cardiac conditions, such as e.g. ischemia, arrhythmias etc.

### SUMMARY OF THE INVENTION

An electrocardiographic system, the electrocardiographic system includes: (i) a processor, adapted to receive three electrocardiographic signals and to provide a cardiological problem indication in response to the three electrocardiographic signals, wherein each of the three electrocardiographic signals is detected between a different pair of electrodes out of a electrode group that consists of three electrodes located on a body of a patient; and (ii) an interface, adapted to transmit the cardiological problem indication received from the processor.

An electrocardiographic system, the electrocardiogram system includes: (i) a processor, adapted to receive electrocardiographic signals and to provide electrocardiographic information in response to the electrocardiographic signals, wherein each of the electrocardiographic signals is detected between a different pair of electrodes out of a electrode group that substantially consists of electrodes located on a body of a patient substantially at the following electrodes connection locations: (a) in the second intercostal space to the right of the sternum, (b) on the intersection of the left medial clavicular line and the fifth intercostal space, (c) on the intersection of the fifth intercostal space and left median-axillar line; and (ii) an interface, adapted to transmit the electrocardiographic information received from the processor.

A method for providing a cardiological problem indication, the method includes: (i) receiving three electrocardiographic signals, wherein each of the three electrocardiographic signals is detected between a different pair of electrodes out of a electrode group that consists of three electrodes located on a body of a patient; and (ii) providing a cardiological problem indication in response to the three electrocardiographic signals.

A method for electrocardiographic detecting, the method includes: (i) receiving electrocardiographic signals, wherein each of the electrocardiographic signals is detected between a different pair of electrodes out of a electrode group that substantially consists of electrodes located on a body of a patient substantially at the following electrodes connection locations: (a) in the second intercostal space to the right of the sternum, (b) on the intersection of the left medial clavicular line and the fifth intercostal space, (c) on the intersection of the fifth intercostal space and left median-axillar line; and (ii) providing electrocardiographic information in response to the electrocardiographic signals.

### BRIEF DESCRIPTION OF THE DRAWINGS

The foregoing and other objects, features, and advantages of the present invention will become more apparent from the following detailed description when taken in conjunction with the accompanying drawings. In the drawings, similar reference characters denote similar elements throughout the different views, in which:

FIGS. 1a and 1b illustrate implementation of Nehb's prior art 3-lead bipolar ECG technique;

FIGS. 2a and 2b illustrate an electrocardiographic system, according to an embodiment of the invention

FIGS. 3a through 3d illustrates a comparison between cardiograms recorded by an embodiment of the electrocardiographic system, and cardiograms recorded using a standard 12 leads ECG;

FIGS. 4a and 4b illustrate a method for electrocardiographic detecting, according to an embodiment of the invention;

FIG. 5 illustrates an electrocardiographic system, according to an embodiment of the invention; and

FIG. 6 illustrates a method for providing a cardiological problem indication, according to an embodiment of the invention.

### DETAILED DESCRIPTION OF THE DRAWINGS

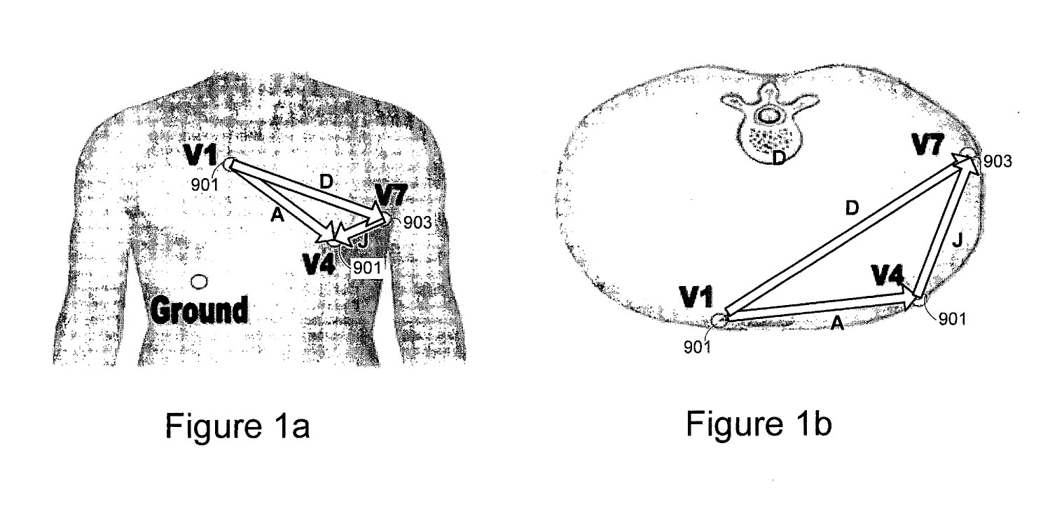

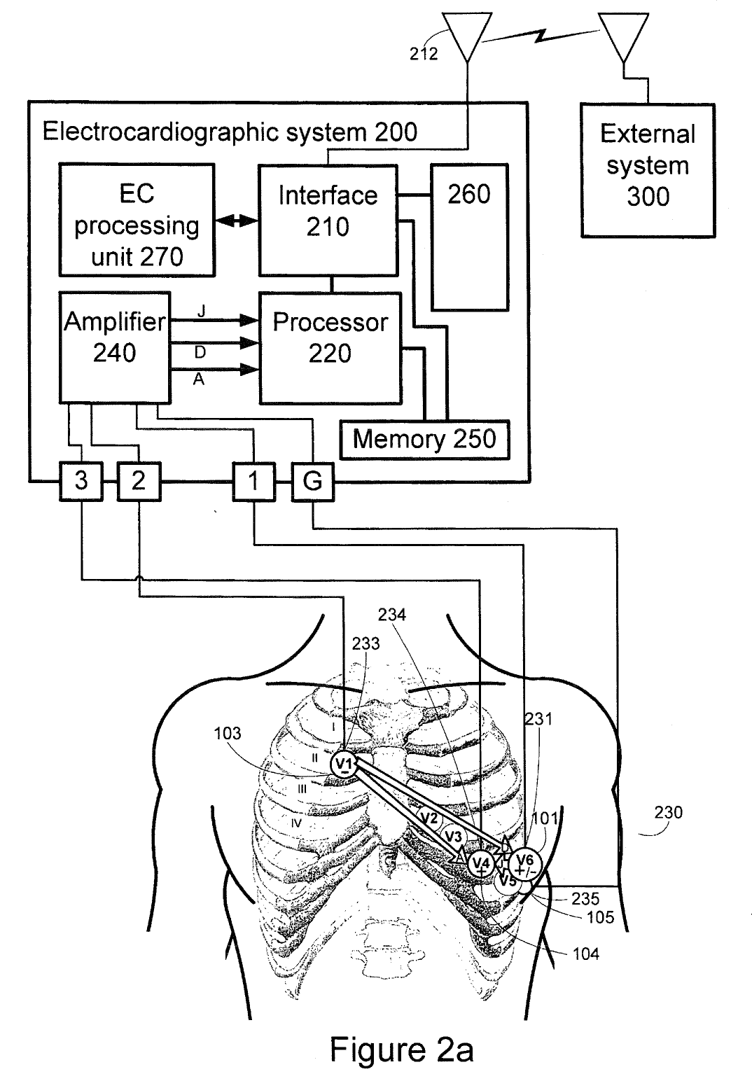

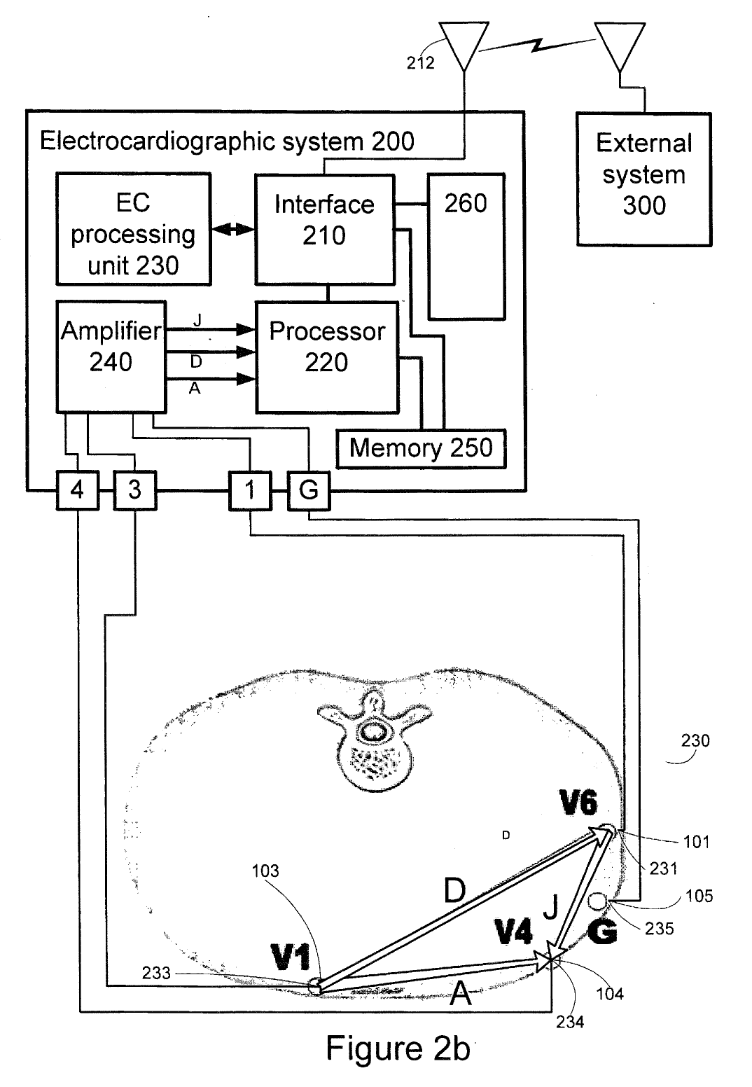

FIGS. 2a and 2b illustrate electrocardiographic system 200, according to an embodiment of the invention wherein FIG. 1a illustrate a coronal view of a mail patient, and FIG. 1b illustrates a transverse cross section thereof. Electrocardiogram system 200 includes processor 220 and interface 210.

Processor 220 is adapted to receive electrocardiographic signals and to provide electrocardiographic information in response to the electrocardiographic signals, wherein each of the electrocardiographic signals is detected between a different pair of electrodes out of an electrode group 230 that substantially consists of electrodes located on a body of a patient substantially at the following electrodes connection locations: (a) in the second intercostal space to the right of the sternum (denoted 103), (b) on the intersection of the left medial clavicular line and the fifth intercostal space (denoted 104), (c) on the intersection of the fifth intercostal space and left median-axillar line (denoted 101). Electrode group 230 can also include one or more ground electrodes.

Referring to accepted electrocardiography 12-leads electrograph electrode connection locations, it is clear to a person who is skilled in the art that: (a) the electrode connection location in the second intercostal space to the right of the sternum is commonly referred to as V1; (b) the electrode connection location on the intersection of the left medial clavicular line and the fifth intercostal space is commonly referred to as V4; and (c) the electrode connection location on the intersection of the fifth intercostal space and left median-axillar line is commonly referred to as V6.

It is noted that electrode group 230 can belong to electrocardiography system 200 or be connected to such a system. The electrodes that form electrode group 230 can be detachably connected to such a system but this is not necessarily so.

Conveniently, the electrode group 230 includes (or is connected to) three signal electrodes (first through third electrodes 231, 233, and 234) and a single ground electrode 235, wherein:

First electrode 231 of electrode group 230 is substantially located on the body of the patient on the intersection of the fifth intercostal space and left median-axillar line (i.e. electrode connection location 101);

Second electrode 233 of electrode group 230 is substantially located on the body of the patient in the second intercostal space to the right of the sternum (i.e. electrode connection location 103);

Third electrode 234 of electrode group 230 is substantially located on the body of the patient on the intersection of the left medial clavicular line and the fifth intercostal space (i.e. electrode connection location 104); and

Ground electrode 235 is substantially located on the body of the patient in proximity to an electrode of electrode group 230 that is located on the intersection of the fifth intercostal space and left median-axillar line (conveniently in proximity to first electrode 231, denoted 105). According to an embodiment of the invention, ground electrode 235 is located below first electrode 231, in the sixth intercostal space.

According to such an embodiment of the invention, the electrocardiographic signals detected are conveniently: (a) a first electrocardiographic signal (also referred to a D-electrocardiographic signal) is detected between second electrode 233 and first electrode 231; (b) a second electrocardiographic signal (also referred to as A-electrocardiographic signal) is detected between second electrode 233 and third electrode 234; and (c) a third electrocardiographic signal (also referred to as J-electrocardiographic signal) is detected between third electrode 234 and first electrode 231. An electrocardiographic signal is detected as the difference between voltage levels detected by a pair of electrodes.

It is noted that, according to an embodiment of the invention, since both the first and the third electrocardiographic signals are detected between first electrode 231 and another electrode, the first and the third electrocardiographic signals are detected at different times.

Further more, according to an embodiment of the invention, first electrode 231 is adapted to serve as a positive electrode during a detecting of the first electrocardiographic signal, and as a negative electrode during a detecting of the third electrocardiographic signal (or vise versa).

It is noted that the first electrocardiographic signal (denoted D) conveniently characterizes a potential of a posterior wall of a left ventricle of a heart of the patient. Similarly, the second electrocardiographic signal (denoted A) conveniently characterizes a potential of an anterior wall of the left ventricle, and the third electrocardiographic signal (denoted J) conveniently characterizes a diaphragmal surface of the heart.

Table 2 illustrates a positioning of electrodes 230, according to an embodiment of the invention.

TABLE 2

| Lead pairs (Nehb adapted) | | D | A | J |
|---|---|---|---|---|
| Red (−) | V1 | X | X | |
| Black (+) | V4 | | X | X |
| Yellow (+/−) | V6 | X | | X |
| Green (G) | Close to V6 | Ground | Ground | Ground |

As aforementioned, first electrode 231 can use for detecting of the first and the third electrocardiographic signals having opposite polarities in each case. For example, according to an embodiment of the invention wherein second electrode 233 serves as a negative terminal and fourth electrode serves as a positive terminal 234 (thus enabling detection of A-electrocardiographic signal), first electrode 231 serves as a positive terminal during a detecting of the first electrocardiographic signal, thus enabling the detection of D-electrocardiographic signal, as a negative terminal during a detecting of the third electrocardiographic signal, thus enabling the detecting of J-electrocardiographic signal.

It is noted that the same could be achieved by an additional fourth electrode (not illustrated) that is located in an immediate proximity to first electrode 231, even though it is conveniently achieved by a single first electrode 231. However, according to such an embodiment of the invention, as first electrode 231 and the fourth electrode need to be located on the body of the patient in immediate proximity to each other, it is noted that according to an embodiment of the invention, first electrode 231 and the fourth are incorporated into an electrodes assembly (not shown), which is adapted to be connected to the body of the patient, and to ensure electrical connectivity of the electrodes incorporated in which to the matching electrodes connection locations, as well as to isolate the electrodes incorporated in the electrodes assembly from each other.

According to different embodiments of the invention, the electrodes assembly includes any subgroup of a group of proximate electrodes that includes first electrode 231, the fourth electrode, and ground electrode 235 (e.g. the electrodes assembly can include first electrode 231 and ground electrode 235).

It is noted that incorporating two or three electrodes into a single electrodes assembly can serve for two purposes. Firstly, as electrocardiographic system 200 could be conveniently used in emergency situation, where time is a crucial factor, not only does it takes less time to connect the electrodes assembly to the body of the patient, it also lessen the likelihood of an electrode misallocated due to panic, poor working conditions, etc. Secondly, a preassembled electrodes assembly facilitates a closer locating of the electrodes that needs to be as proximate as possible, thus increasing the accuracy of the electrocardiographic measurement.

It is noted that, conveniently, electrocardiographic system 200 is further adapted to detect at least one of the electrocardiographic signals by implementing ground electrode 235 that is substantially located in proximity to first electrode 231 (i.e. in the proximity of electrode connection location 101). It is noted that according to other embodiments of the invention, ground electrode 235 is located elsewhere on the body of the patient, such as (though not necessarily) in proximity to second electrode 233 or in proximity to third electrode 234, wherein an appropriate electrodes assembly could incorporate the ground electrode as well as one of the third and the fourth electrodes 233 and 234.

Conveniently, the connection location of ground electrode 235 to the body of the patient is less significant than those of the other electrodes. The connection location of ground electrodes 235 may be determined in response to a convenience of connection ground electrode 235 to the body of the patient, to facilitate quick connection of the electrodes of the electrode group 230, and hence quick detecting of the electrocardiographic signals.

It is noted that, according to an embodiment of the invention, electrocardiographic system 200 further includes amplifier 240, that is adapted to amplify at least one of the electrocardiographic signals prior to the receiving of the at least one of the electrocardiographic signals by processor 220.

Referring again to the aforementioned interface 210, interface 210 is adapted to transmit the electrocardiographic information received from processor 220. According to different embodiments of the invention, interface 210 is adapted to transmit the electrocardiographic information to a unit of electrocardiographic system 200, to external system 300 (or a unit thereof), or to both, wherein the electrocardiographic information conveniently requires further processing to provide electrocardiographically assessable information.

According to an embodiment of the invention, electrocardiographic system 200 further includes electrocardiographic processing unit 270 that is adapted to process the electrocardiographic information received from processor 220, to provide electrocardiographically assessable information.

According to an embodiment of the invention, interface 210 includes an external system interface (not shown) for the providing of the electrocardiographic information to external system 300, wherein the providing of the electrocardiographic information is provided wither via a data cable (not shown) or wirelessly (as illustrated in FIG. 1, e.g. via antenna 212). According to an embodiment of the invention, interface 210 is adapted to provide the electrocardiographic information wirelessly.

It is noted that, according to an embodiment of the invention wherein electrocardiographic system 200 includes electrocardiographic processing unit 270, interface may provide to external system 300 the electrocardiographically assessable information instead of (or on top of) the electrocardiographic information.

According to an embodiment of the invention, external system 300 is located in proximity to electrocardiographic system 200, such as when external system 300 is part of a medical emergency kit (e.g. of an ambulance etc.), or when external system 300 is adapted for the displaying of the electrocardiographic information (or the electrocardiographic assessable information), or for the printing thereof.

According to another embodiment of the invention, external system 300 is a distant external system that can reside in a hospital or in emergencies support center, and is adapted to provide the electrocardiographic information (or the electrocardiographic assessable information) to an ECG professional, for immediate assessing of the provided information. According to such an embodiment of the invention, the connection between electrocardiographic system 200 and external system 300 is conveniently a wireless one (e.g. supported by cellular telephony communication).

According to an embodiment of the invention, electrocardiographic system 200 is a compact mobile electrocardiographic system, such as a one that could be included in an emergency medical kit, or which could be stored by the patient that suffers from a sever heart condition.

Conveniently, electrocardiographic system 200 is designed for usage as a compact portable cardiographer device for express diagnostics in the situations when professional cardiological assistance is not available. Being adapted to, according to some embodiments of the invention, provide a telecommunication feature that allows a fast transferring of the recorded cardiograms to the professionals allows for faster and more adequate outpatient response in the case of emergencies.

For similar reasons, according to an embodiment of the invention, electrocardiographic system 200 further includes electrodes of electrode group 230, wherein each of the electrodes of the electrode group 230 is adapted to be detachably attached by the patient to the body of the patient at one of the electrode connection locations.

According to an embodiment of the invention, electrocardiographic system 200 further includes memory unit 250, for storing of the electrocardiographic information (or the electrocardiographic assessable information), or part of which.

It is noted that, according to an embodiment of the invention, electrocardiographic system 200 is adapted to detect the electrocardiographic signals of the patient in a normal condition and storing the relative electrocardiographic information in memory unit 250. Later, in situation of emergency, the previously stored electrocardiographic information pertaining to the normal condition could be provided by electrocardiographic system 200 along with the currently provided electrocardiographic information pertaining to the situation of emergency, thus offering an ECG professional more information upon which to analyze the provided electrocardiographic information.

According to an embodiment of the invention, electrocardiographic system 200 further includes display 260, for displaying the electrocardiographic information (or the electrocardiographic assessable information), or part of which.

It is clear to a person who is skilled in the art that the modification of the method of classic Nehb's electrode placing that is disclosed according to the herein offered invention includes placing of one or more electrodes in V6 or the immediate proximity thereof, instead of in V7. Another modification disclosed is a locating of ground electrode 235 aside a V6 electrode. Both of those modifications facilitate the implementing of electrocardiographic system 200 as a portable electrocardiographer.

It is noted that the topographic approximation of ground electrode 235 to first electrode 231 will not interfere with the quality and capability to be evaluate of the ECG recordings, since the direction of the leads vectors is preserved; on the other hand it will be actually of benefit when used in an electrocardiographic system 200 that is designed for emergency diagnostics of life threatening heart conditions when the rapid, reliable and simple method of ECG recording is critical.

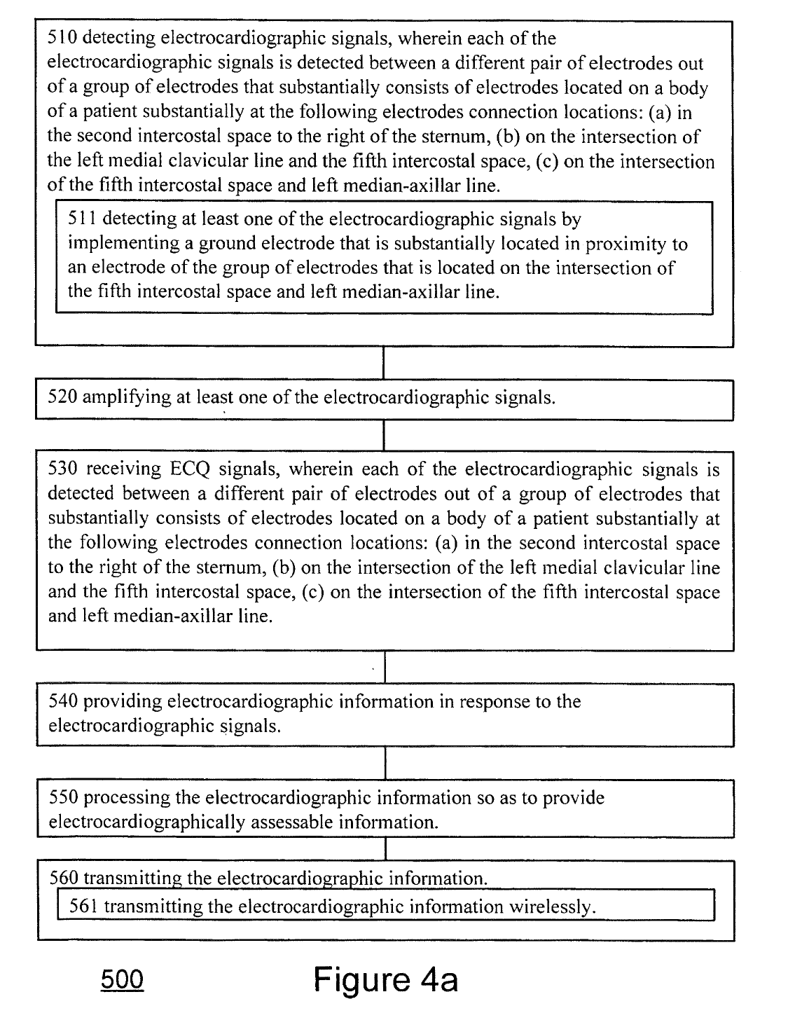

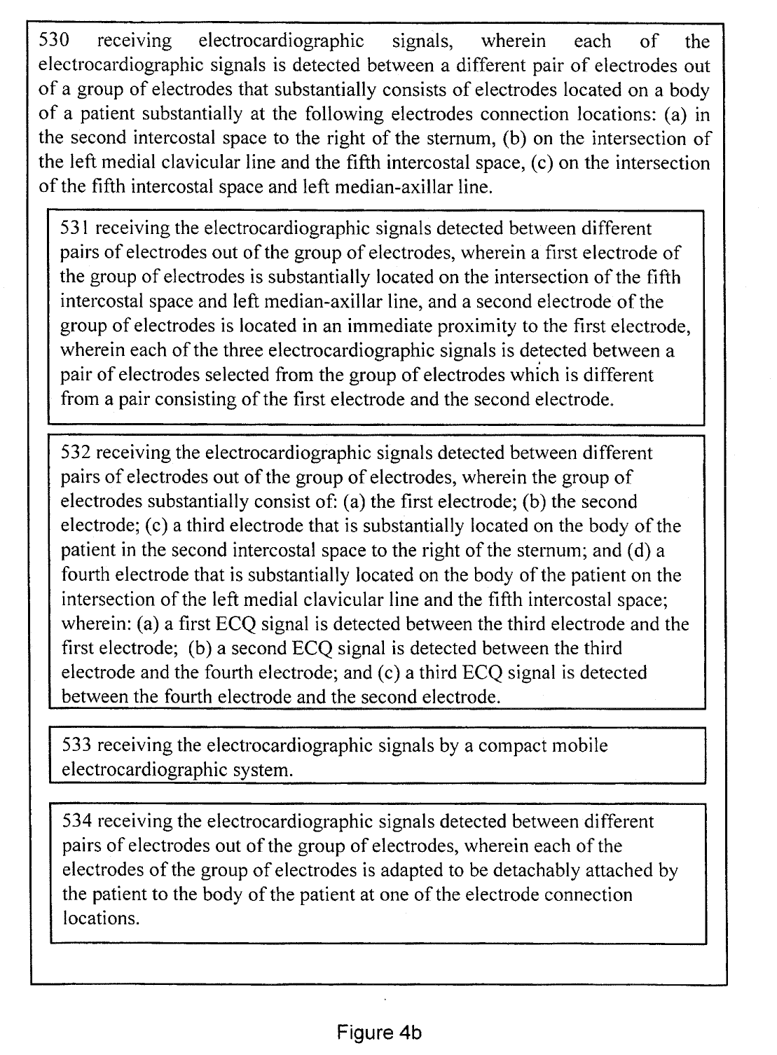

FIGS. 4a and 4b illustrates method 500 for electrocardiographic detecting, according to an embodiment of the invention, wherein FIG. 4b illustrates different implementations of the stage of receiving, according to different embodiments of invention. It is noted that conveniently, different embodiments of method 500 are adapted to be carried out by different embodiments of electrocardiographic system 200, and as is clear to a person who is skilled in the art, method 500 could be easily adapted to support the different embodiments of electrocardiographic system 200 (some of which are discussed above), even if not specifically discussed in relation to method 500. Therefore, a person who is skilled in the art may benefit from considering method 500 and different embodiments thereof in view of the different discussed embodiments of electrocardiographic system 200.

According to an embodiment of the invention, method 500 starts with stage 510 of detecting electrocardiographic signals, wherein each of the electrocardiographic signals is detected between a different pair of electrodes out of an electrode group that substantially consists of electrodes located on a body of a patient substantially at the following electrodes connection locations: (a) in the second intercostal space to the right of the sternum, (b) on the intersection of the left medial clavicular line and the fifth intercostal space, (c) on the intersection of the fifth intercostal space and left median-axillar line.

According to an embodiment of the invention, stage 510 includes stage 511 of detecting at least one of the electrocardiographic signals by implementing a ground electrode that is substantially located in proximity to an electrode of the electrode group that is located on the intersection of the fifth intercostal space and left median-axillar line.

According to an embodiment of the invention, method 500 includes stage 520 of amplifying at least one of the electrocardiographic signals. Referring to the examples set forward in the previous drawings, stage 520 is conveniently carried out by amplifier 240.

Method 500 continues with stage 530 of receiving electrocardiographic signals, wherein each of the electrocardiographic signals is detected between a different pair of electrodes out of a electrode group that substantially consists of electrodes located on a body of a patient substantially at the following electrodes connection locations: (a) in the second intercostal space to the right of the sternum, (b) on the intersection of the left medial clavicular line and the fifth intercostal space, (c) on the intersection of the fifth intercostal space and left median-axillar line. Referring to the examples set forward in the previous drawings, stage 530 is conveniently carried out by processor 220.

According to an embodiment of the invention, stage 530 includes stage 531 of receiving the electrocardiographic signals detected between different pairs of electrodes out of the electrode group, wherein a first electrode of the electrode group is substantially located on the intersection of the fifth intercostal space and left median-axillar line, and a fourth electrode of the electrode group is located in an immediate proximity to the first electrode, wherein each of the three electrocardiographic signals is detected between a pair of electrodes selected from the electrode group which is different from a pair consisting of the first electrode and the fourth electrode. However, as disclosed above, conveniently there is no need in two electrodes, and the first electrode suffices, wherein—as aforementioned—the first electrodes may serve as a terminal having a first polarity (e.g. positive) during the detecting of the first electrocardiographic signal, and as a terminal having a second polarity (e.g. negative) during the detecting of the third signal.

According to an embodiment of the invention, stage 530 includes stage 532 of receiving the electrocardiographic signals detected between different pairs of electrodes out of the electrode group, wherein the electrode group substantially consist of: (a) a first electrode that is substantially located on the intersection of the fifth intercostal space and left median-axillar line; (b) a second electrode that is substantially located on the body of the patient in the second intercostal space to the right of the sternum; and (c) a third electrode that is substantially located on the body of the patient on the intersection of the left medial clavicular line and the fifth intercostal space; wherein: (a) a first electrocardiographic signal is detected between the second electrode and the first electrode; (b) a second electrocardiographic signal is detected between the second electrode and the third electrode; and (c) a third electrocardiographic signal is detected between the third electrode and the first electrode.

According to an embodiment of the invention, stage 530 includes stage 533 of receiving the electrocardiographic signals by a compact mobile electrocardiographic system.

According to an embodiment of the invention, stage 530 includes stage 534 of receiving the electrocardiographic signals detected between different pairs of electrodes out of the electrode group, wherein each of the electrodes of the electrode group is adapted to be detachably attached by the patient to the body of the patient at one of the electrode connection locations.

Stage 530 is followed by stage 540 of providing electrocardiographic information in response to the electrocardiographic signals. It is noted that, according to an embodiment of the invention, stage 540 includes providing the electrocardiographic information by the compact mobile electrocardiographic system. Referring to the examples set forward in the previous drawings, stage 540 is conveniently carried out by processor 220.

According to an embodiment of the invention, stage 540 is followed by stage 550 of processing the electrocardiographic information to provide electrocardiographically assessable information. Referring to the examples set forward in the previous drawings, stage 540 is carried out, according to an embodiment of the invention, by electrocardiographic processing unit 270.

Method 500 continues with stage 560 of transmitting the electrocardiographic information. It is noted that, according to some embodiments of the invention, the transmitting may include transmitting the electrocardiographically assessable information in stead of (or on top of) the electrographic information. Referring to the examples set forward in the previous drawings, stage 560 is conveniently carried out by interface 210.

According to an embodiment of the invention, stage 560 includes stage 561 of transmitting the electrocardiographic information wirelessly.

According to an embodiment of the invention, stage 560 includes transmitting the electrocardiographic information by the compact mobile electrocardiographic system.

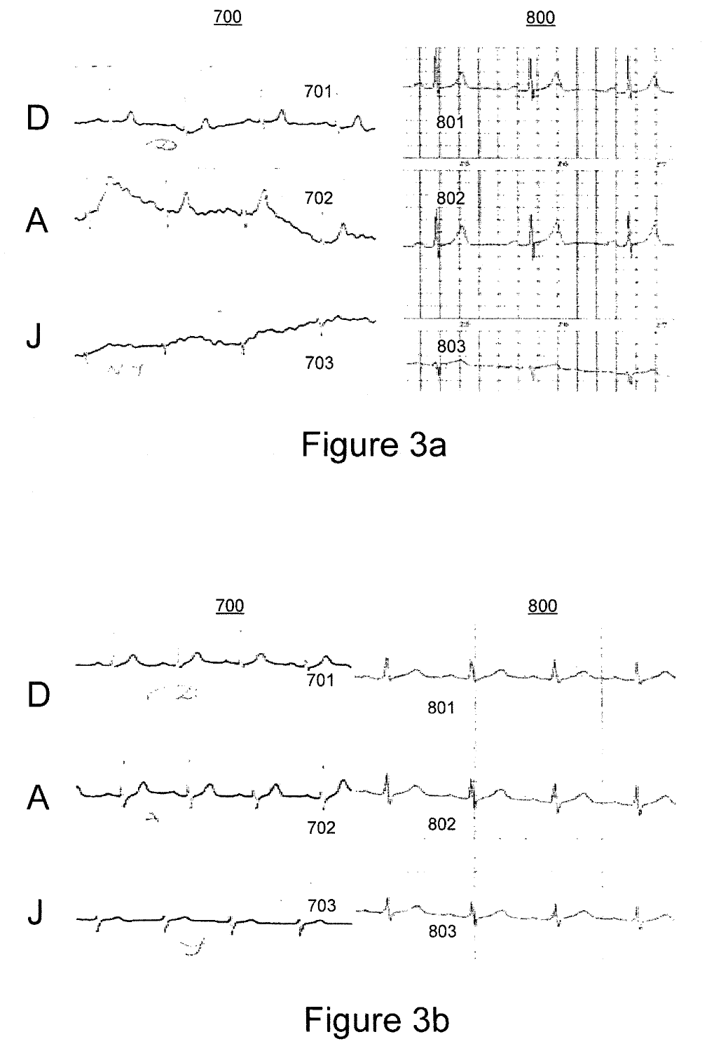

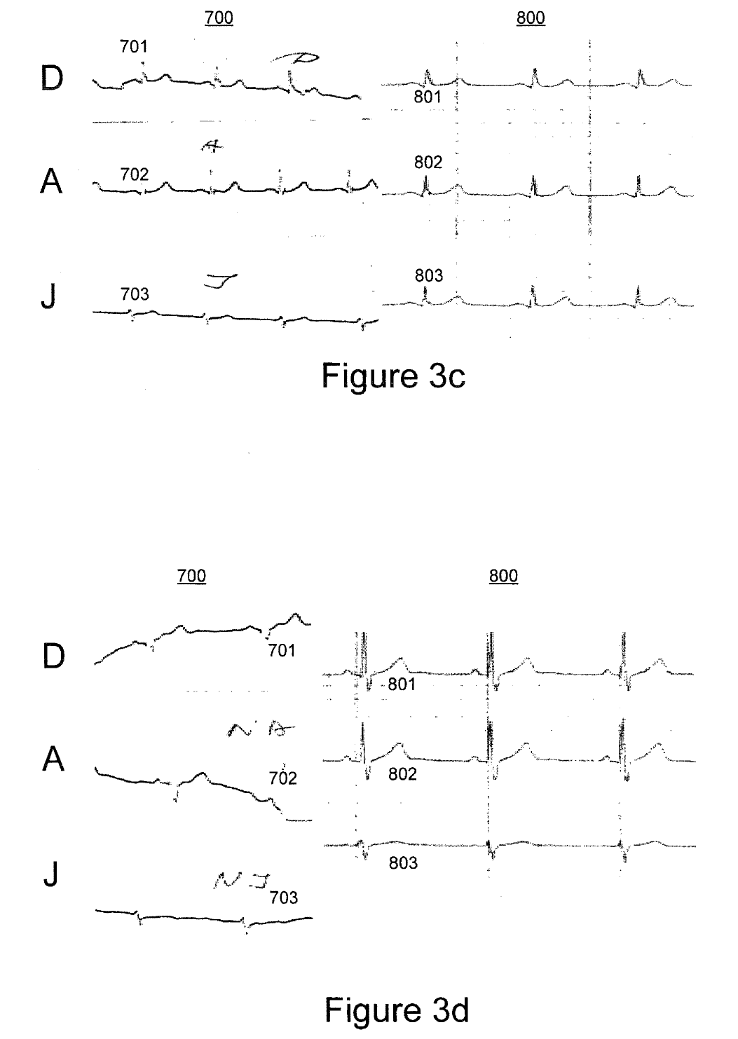

FIGS. 3a through 3d illustrates a comparison between cardiograms 800 recorded by an embodiment of electrocardiographic system 200, and cardiograms 700 recorded using a standard 12 leads ECG, wherein the electrocardiographic signals illustrated therefor are the classic Nehb's electrocardiographic signals Nehb's A, Nehb's D, and Nehb's J, referred to in the background.

It is noted that in each of FIGS. 3a through 3d, the topmost cardiograms are cardiogram 801 which is a D-electrocardiographic signal, and cardiogram 701 that is a Nehb's D-electrocardiographic signal; the middle cardiograms are cardiogram 802 which is an A-electrocardiographic signal, and cardiogram 702 that is a Nehb's A-electrocardiographic signal; and the bottom cardiograms are cardiogram 803 which is a J-electrocardiographic signal, and cardiogram 703 that is a Nehb's J-electrocardiographic signal, wherein each of the FIGS. 3a through 3d illustrates cardiograms detected for a different healthy patient.

A person who is skilled in the art would learn that the results illustrated in FIGS. 3a through 3d indicate a very good correlation between the ECG cardiograms recorded by a standard cardiographer with the ECG cardiograms recorded by an embodiment of electrocardiographic system 200, using the herein disclosed adapted Nehb's leads. It is noted that these observations agree with previous prior-art studies (e.g. Seeberger M. D., et al. (1997) “The Inverse Nehb J lead increases the sensitivity of Holter Electrocardiographic monitoring for detecting myocardial ischemia.” Am. J. Cardiol 80:1-5; and Osterhues, H.-H., et al. (1994) “Improved detection of transient myocardial ischemia by a new lead combination: value of bipolar lead Nehb D for Holter-monitoring.” Am. Heart J. 127:559-566).

As will be clear to a person who is skilled in the art, there is a practical identity of the ECG cardiograms recorded by electrocardiographic system 200 and those recorded by the standard 12-leads ECG, and presence of sufficient number of diagnostic criteria as compared to the 12-lead ECG, which allow an unambiguously determining of most of ECG parameters and diagnostic criteria.

It is by now clear to a person who is skilled in the art that the electrographic information provided by electrocardiographic system 200 proved to be very applicable and informative for ECG diagnostics, providing good correlation with standard 12 lead ECG but requiring less time for recording, as only three active leads are in use.

Being positioned on the chest in the immediate proximity to the heart, and being aligned to the anatomic position of the heart, the herein disclosed leads are very sensitive and allow accurate diagnostics of different heart conditions. Furthermore, all the active leads according to the disclosed invention are located on an anterolateral plane of a chest wall of the patient, thus requiring little anatomical window, hence making the herein disclosed technique convenient for express diagnostics of different heart conditions, including emergency cases, such as acute ischemia or arrhythmias.

Adaptation of Nehb's leads as described herein suggests several applications for ECG diagnostics. Especially valuable is the implementation of the herein disclosed systems and methods for compact cardiographers. Adapted Nehb's leads make ECG recording simple and fast, require a small anatomic window, while still allow to obtain complete cardiographic criteria necessary for the express diagnostics in the cases when the patients are far from the inpatient hospitals and it is complicated to receive fast cardiological assistance.

By way of example, electrocardiographic systems 200 that are compact cardiographers of such kind can be located in dentists' offices, surgeons, gynecologists, etc. and to be used for other situations when the professional cardiological help is for any reason delayed.

According to another embodiment of the invention, the systems and methods herein disclosed can be utilized for a relatively simple self-control for chronic patients with cardiological history. By all means, timely ECG diagnostics will undoubtedly increase the chances for successful medical help to the patient in acute situations.

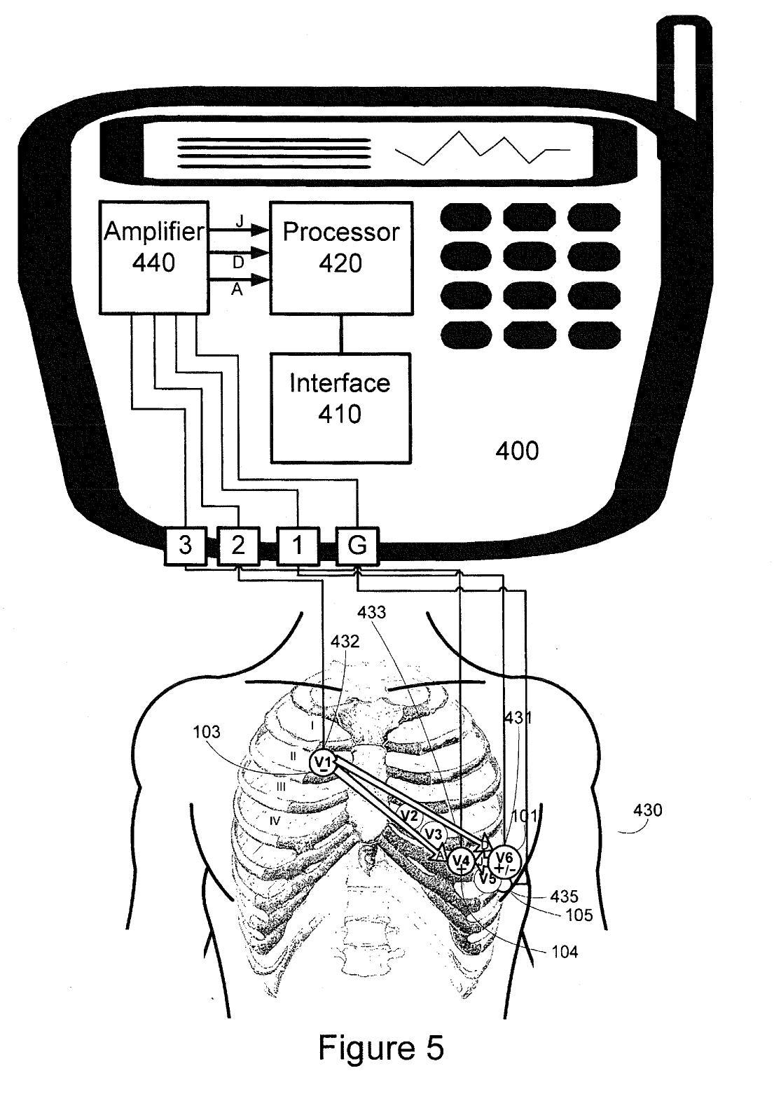

FIG. 5 illustrates electrocardiographic system 400, according to an embodiment of the invention. Electrocardiographic system 400 includes: (a) processor 420 that is adapted to receive three electrocardiographic signals and to provide a cardiological problem indication in response to the three electrocardiographic signals, wherein each of the three electrocardiographic signals is detected between a different pair of electrodes out of electrode group 430 that consists of three electrodes 431, electronic messages generating system integrated agent 432 and 433 located on a body of a patient; and (b) interface 410, adapted to transmit the cardiological problem indication received from processor 420.

According to an embodiment of the invention, processor 420 is adapted to provide the cardiological problem indication in response to the three electrocardiographic signals, wherein each of the three electrocardiographic signals is detected between a different pair of electrodes out of electrode group 430 that substantially consists of three electrodes 431, electronic messages generating system integrated agent 432 and 433 located on a body of a patient substantially at the following electrodes connection locations: (a) in the second intercostal space to the right of the sternum, (b) on the intersection of the left medial clavicular line and the fifth intercostal space, (c) on the intersection of the fifth intercostal space and left median-axillar line.

According to an embodiment of the invention, system 400 is further adapted to detect at least one of the electrocardiographic signals by implementing ground electrode 435 that is, according to an embodiment of the invention, substantially located in proximity to an electrode of electrode group 430 that is located on the intersection of the fifth intercostal space and left median-axillar line.

According to an embodiment of the invention, processor 420 is further adapted to provide electrocardiographic information in response to the three electrocardiographic signals (similarly to system 200 disclosed above). According to an embodiment of the invention, processor 420 is adapted to process the electrocardiographic information, to provide the cardiological problem indication.

According to an embodiment of the invention, interface 410 is adapted to transmit the cardiological problem indication wirelessly.

According to an embodiment of the invention, system 400 further includes amplifier 440 that is adapted to amplify at least one of the electrocardiographic signals prior to the receiving of the at least one of the electrocardiographic signals by processor 420.

According to an embodiment of the invention, electrocardiographic system 400 is a compact mobile electrocardiographic system, which is conveniently either handily portable by a medical practitioner or a first aid provider, in order to quickly diagnose a patient, or adapted to be carried by a patient in potential hazard for longer periods of time, either for analysis or for monitoring his condition. Being able to transport the data wirelessly facilitate continuing monitoring by a medical center.

According to an embodiment of the invention, processor 420 is adapted to provide an acute arrhythmias cardiological problem indication in response to the three electrocardiographic signals.

According to an embodiment of the invention, processor 420 is adapted to provide an ischemia cardiological problem indication in response to the three electrocardiographic signals.

According to an embodiment of the invention, interface 410 is adapted to transmit the cardiological problem indication to a handheld communication device, e.g. a cellular phone, a lap-top computer, or a personal digital assistant.

According to an embodiment of the invention, electrocardiographic system 400 further includes first, second and third electrodes 431, electronic messages generating system integrated agent 432 and 433 (and potentially also ground electrode 435), wherein, according to an embodiment of the invention, the three electrodes 431, 432 and 433 (and potentially also ground electrode 435) are adapted to be self attached to a body of a patient by the patient himself.

Thus, a patient who is in a potential hazard can be easily trained to connect the electrodes of system 400 to his body, either for regular monitoring, or when the patient feels (or have other reason to suspect) he may suffer from a hazardous cardiological incident. Limiting the number of electrodes to four (counting ground electrode 435 as well), comparing to five electrodes which are needed for example by Holter devices, facilitates a faster connecting of the electrodes to the body of the patient (either by the patient or by other person), and ease the training required for the correct connecting of the electrodes, thus enabling a chronic cardiological patient to be trained to place the electrodes autonomously, facilitating the use of electrocardiographic system 400 as a self-usable electrocardiographic system, adapted to be owned and operated by people suffering from a heart condition.

It would be clear to a person who is skilled in the art that other components and abilities disclosed in relation to system 200 and to different embodiments thereof could also be applied to system 400 and to different embodiments thereof, mutatis mutandis.

It is noted that conveniently, system 400 and different embodiments thereof are adapted to carry out method 900 disclosed below, and different embodiments thereof.

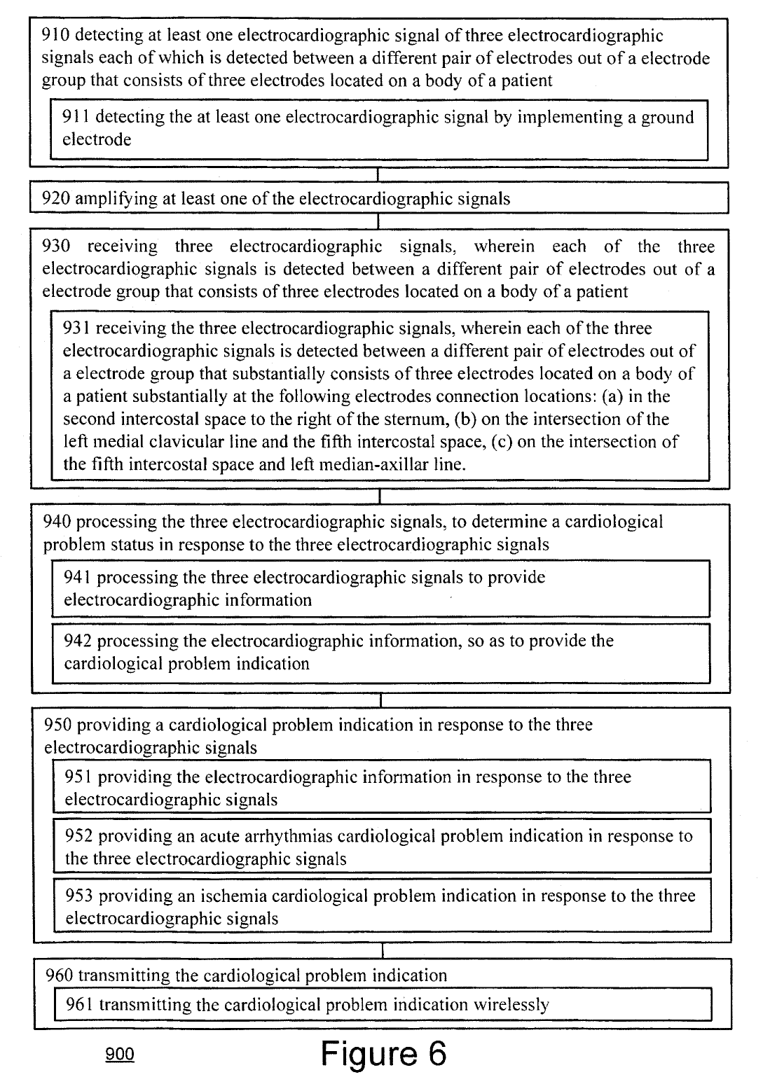

FIG. 6 illustrates method 900 for providing a cardiological problem indication, according to an embodiment of the invention.

According to an embodiment of the invention, method 900 starts with stage 910 of detecting at least one electrocardiographic signal of three electrocardiographic signals each of which is detected between a different pair of electrodes out of an electrode group that consists of three electrodes located on a body of a patient.

According to an embodiment of the invention, stage 910 includes stage 911 of detecting the at least one electrocardiographic signal by implementing a ground electrode. According to an embodiment of the invitation, the ground electrode is substantially located in proximity to an electrode of the electrode group that is located on the intersection of the fifth intercostal space and left median-axillar line.

According to an embodiment of the invention, stage 910 is followed by stage 920 of amplifying at least one of the electrocardiographic signals.

Method 900 continues with stage 930 of receiving three electrocardiographic signals, wherein each of the three electrocardiographic signals is detected between a different pair of electrodes out of an electrode group that consists of three electrodes located on a body of a patient.

According to an embodiment of the invention, stage 930 includes stage 931 of receiving the three electrocardiographic signals, wherein each of the three electrocardiographic signals is detected between a different pair of electrodes out of a electrode group that substantially consists of three electrodes located on a body of a patient substantially at the following electrodes connection locations: (a) in the second intercostal space to the right of the sternum, (b) on the intersection of the left medial clavicular line and the fifth intercostal space, (c) on the intersection of the fifth intercostal space and left median-axillar line.

Stage 930 is conveniently followed by stage 940 of processing the three electrocardiographic signals, to determine a cardiological problem status in response to the three electrocardiographic signals.

According to an embodiment of the invention, stage 940 further includes stage 941 of processing the three electrocardiographic signals to provide electrocardiographic information (see for example discussion in relation to system 200 and to method 500).

According to an embodiment of the invention, stage 940 includes stage 942 of processing the electrocardiographic information, to provide the cardiological problem indication.

Method 900 continues with stage 950 of providing a cardiological problem indication in response to the three electrocardiographic signals. Conveniently, the cardiological problem indication is provided only if the cardiological problem status was determined problematic after processing the three electrocardiographic signals. It is noted that processing the three electrocardiographic signals suffices to detect a wide variety of cardiological problems, an early detection of which (that hopefully leads to a short door to needle span) may save the life of the patient, or significantly limit any damages of different cardiological situations.

According to an embodiment of the invention, stage 950 includes stage 951 of providing the electrocardiographic information in response to the three electrocardiographic signals (wherein the electrocardiographic information is conveniently acquired during stage 941).

According to an embodiment of the invention, stage 950 includes stage 952 of providing an acute arrhythmias cardiological problem indication in response to the three electrocardiographic signals.

According to an embodiment of the invention, stage 950 includes stage 953 of providing an ischemia cardiological problem indication in response to the three electrocardiographic signals.

Stage 950 is conveniently followed by stage 960 of transmitting the cardiological problem indication.

According to an embodiment of the invention, stage 960 further includes transmitting the electrocardiographic information (wherein it is noted that if no cardiological problem indication is generated, the electrocardiographic information could be transmitting separately).

According to an embodiment of the invention, stage 960 includes stage 961 of transmitting the cardiological problem indication wirelessly.

According to an embodiment of the invention, stage 960 includes transmitting the cardiological problem indication to a handheld communication device.

It is noted that according to an embodiment of the invention, the stages of receiving and providing (and potentially all the stages of method 900) are carried out by a compact mobile electrocardiographic system.

It would be clear to a person who is skilled in the art that other stages and details disclosed in relation to method 500 and to different embodiments thereof could also be applied to method 900 and to different embodiments thereof, mutatis mutandis.

It is noted that conveniently, method 900 and different embodiments thereof are adapted to be carry out by system 400 disclosed below, and different embodiments thereof.

The present invention can be practiced by employing conventional tools, methodology, and components. Accordingly, the details of such tools, components, and methodology are not set forth herein in detail. In the previous descriptions, numerous specific details are set forth, in order to provide a thorough understanding of the present invention. However, it should be recognized that the present invention might be practiced without resorting to the details specifically set forth.

Only exemplary embodiments of the present invention and but a few examples of its versatility are shown and described in the present disclosure. It is to be understood that the present invention is capable of use in various other combinations and environments and is capable of changes or modifications within the scope of the inventive concept as expressed herein.

We claim:

1. An electrocardiographic system, the electrocardiogram system comprises:
a processor, adapted to receive electrocardiographic signals and to provide electrocardiographic information in response to the electrocardiographic signals, wherein each of the electrocardiographic signals is detected between a different pair of electrodes out of a electrode group that substantially consists of electrodes located on a body of a patient substantially at the following electrodes connection locations: (a) in the second intercostal space to the right of the sternum, (b) on the intersection of the left medial clavicular line and the fifth intercostal space, (c) on the intersection of the fifth intercostal space and left median-axillar line; and
an interface, adapted to transmit the electrocardiographic information received from the processor.

2. The electrocardiographic system according to claim 1, wherein the electrode group substantially consist of: (a) a first electrode that is substantially located on the intersection of the fifth intercostal space and left median-axillar line; (b) a second electrode that is substantially located on the body of the patient in the second intercostal space to the right of the sternum; and (c) a third electrode that is substantially located on the body of the patient on the intersection of the left medial clavicular line and the fifth intercostal space;
wherein: (a) a first electrocardiographic signal is detected between the second electrode and the first electrode; (b) a second electrocardiographic signal is detected between the second electrode and the third electrode; and (c) a third electrocardiographic signal is detected between the third electrode and the first electrode.

3. The electrocardiographic system according to claim 1, further adapted to detect at least one of the electrocardiographic signals by implementing a ground electrode that is substantially located in proximity to an electrode of the electrode group that is located on the intersection of the fifth intercostal space and left median-axillar line.

4. The electrocardiographic system according to claim 1, further comprising an electrocardiographic processing unit, that is adapted to process the electrocardiographic information received from the processor, so as to provide electrocardiographically assessable information.

5. The electrocardiographic system according to claim 1, wherein the interface is adapted to provide the electrocardiographic information wirelessly.

6. The electrocardiographic system according to claim 1, further comprising an amplifier that is adapted to amplify at least one of the electrocardiographic signals prior to the receiving of the at least one of the electrocardiographic signals by the processor.

7. The electrocardiographic system according to claim 1, wherein the electrocardiographic system is a compact mobile electrocardiographic system.

8. The electrocardiographic system according to claim 1, further comprising the electrodes of the electrode group, wherein each of the electrodes of the electrode group is adapted to be detachably attached by the patient to the body of the patient at one of the electrode connection locations.

9. A method for electrocardiographic detecting, the method comprises:
receiving electrocardiographic signals, wherein each of the electrocardiographic signals is detected between a different pair of electrodes out of a electrode group that substantially consists of electrodes located on a body of a patient substantially at the following electrodes connection locations: (a) in the second intercostal space to the right of the sternum, (b) on the intersection of the left medial clavicular line and the fifth intercostal space, (c) on the intersection of the fifth intercostal space and left median-axillar line; and
providing electrocardiographic information in response to the electrocardiographic signals.

10. The method according to claim 9, wherein the receiving comprises receiving the electrocardiographic signals detected between different pairs of electrodes out of the electrode group, wherein the electrode group substantially consist of: (a) a first electrode that is substantially located on the intersection of the fifth intercostal space and left median-axillar line; (b) a third electrode that is substantially located on the body of the patient in the second intercostal space to the right of the sternum; and (c) fourth electrode that is substantially located on the body of the patient on the intersection of the left medial clavicular line and the fifth intercostal space; wherein: (a) a first electrocardiographic signal is detected between the third electrode and the first electrode; (b) a second electrocardiographic signal is detected between the third electrode and the fourth electrode; and (c) a third electrocardiographic signal is detected between the fourth electrode and the second electrode.

11. The method according to claim 9, wherein the receiving follows a stage of detecting the electrocardiographic signals, wherein the detecting further comprises detecting at least one of the electrocardiographic signals by implementing a ground electrode that is substantially located in proximity to an electrode of the electrode group that is located on the intersection of the fifth intercostal space and left median-axillar line.

12. The method according to claim 9, further comprising processing the electrocardiographic information so as to provide electrocardiographically assessable information.

13. The method according to claim 9, wherein the transmitting comprises transmitting the electrocardiographic information wirelessly.

14. The method according to claim 9, wherein the receiving follows a stage of amplifying at least one of the electrocardiographic signals.

15. The method according to claim 9, wherein the receiving comprises receiving the electrocardiographic signals by a compact mobile electrocardiographic system; wherein the providing comprises providing the electrocardiographic information by the compact mobile electrocardiographic system; and wherein the transmitting comprises transmitting the electrocardiographic information by the compact mobile electrocardiographic system.

16. The method according to claim 9, wherein the receiving comprises receiving the electrocardiographic signals detected between different pairs of electrodes out of the electrode group, wherein each of the electrodes of the electrode group is adapted to be detachably attached by the patient to the body of the patient at one of the electrode connection locations.

17. An electrocardiographic system, the electrocardiographic system comprises:
a processor, adapted to receive three electrocardiographic signals and to provide a cardiological problem indication in response to the three electrocardiographic signals, wherein each of the three electrocardiographic signals is detected between a different pair of electrodes out of a electrode group that consists of three electrodes located on a body of a patient; and
an interface, adapted to transmit the cardiological problem indication received from the processor.

18. The electrocardiographic system according to claim 17, wherein the processor, is adapted to provide the cardiological problem indication in response to the three electrocardiographic signals, wherein each of the three electrocardiographic signals is detected between a different pair of electrodes out of a electrode group that substantially consists of three electrodes located on a body of a patient substantially at the following electrodes connection locations: (a) in the second intercostal space to the right of the sternum, (b) on the intersection of the left medial clavicular line and the fifth intercostal space, (c) on the intersection of the fifth intercostal space and left median-axillar line.

19. The electrocardiographic system according to claim 17, further adapted to detect at least one of the electrocardiographic signals by implementing a ground electrode.

20. The electrocardiographic system according to claim 17, wherein the processor is further adapted to provide electrocardiographic information in response to the three electrocardiographic signals.

21. The electrocardiographic system according to claim 20, wherein the processor is adapted to process the electrocardiographic information, to provide the cardiological problem indication.

22. The electrocardiographic system according to claim 17, wherein the interface is adapted to transmit the cardiological problem indication wirelessly.

23. The electrocardiographic system according to claim 17, further comprising an amplifier that is adapted to amplify at least one of the electrocardiographic signals prior to the receiving of the at least one of the electrocardiographic signals by the processor.

24. The electrocardiographic system according to claim 17, wherein the electrocardiographic system is a compact mobile electrocardiographic system.

25. The electrocardiographic system according to claim 17, wherein the processor is adapted to provide an acute arrhythmias cardiological problem indication in response to the three electrocardiographic signals.

26. The electrocardiographic system according to claim 17, wherein the processor is adapted to provide an ischemia cardiological problem indication in response to the three electrocardiographic signals.

27. The electrocardiographic system according to claim 17, wherein the interface is adapted to transmit the cardiological problem indication to a handheld communication device.

28. The electrocardiographic system according to claim 17, further comprising the three electrodes which are adapted to be self attached to a body of a patient by the patient himself.

29. A method for providing a cardiological problem indication, the method comprises:
receiving three electrocardiographic signals, wherein each of the three electrocardiographic signals is detected between a different pair of electrodes out of an electrode group that consists of three electrodes located on a body of a patient;
and providing a cardiological problem indication in response to the three electrocardiographic signals.

30. The method according to claim 29, wherein the receiving comprises receiving the three electrocardiographic signals, wherein each of the three electrocardiographic signals is detected between a different pair of electrodes out of a electrode group that substantially consists of three electrodes located on a body of a patient substantially at the following electrodes connection locations: (a) in the second intercostal space to the right of the sternum, (b) on the intersection of the left medial clavicular line and the fifth intercostal space, (c) on the intersection of the fifth intercostal space and left median-axillar line.

31. The method according to claim 29, further comprising detecting at least one of the electrocardiographic signals by implementing a ground electrode.

32. The method according to claim 29, further comprising providing electrocardiographic information in response to the three electrocardiographic signals.

33. The method according to claim 32, wherein the providing of the cardiological problem indication is preceded by processing the electrocardiographic information, to provide the cardiological problem indication.

34. The method according to claim 29, further comprising transmitting the cardiological problem indication wirelessly.

35. The method according to claim 29, wherein the providing of the cardiological problem indication is preceded by amplifying at least one of the electrocardiographic signals.

36. The method according to claim 29, wherein the stages of receiving and providing are carried out by a compact mobile electrocardiographic system.

37. The method according to claim 29, wherein the providing of the cardiological problem indication comprises providing an acute arrhythmias cardiological problem indication in response to the three electrocardiographic signals.

38. The method according to claim 29, wherein the providing of the cardiological problem indication comprises providing an ischemia cardiological problem indication in response to the three electrocardiographic signals.

39. The method according to claim 29, wherein further comprising transmitting the cardiological problem indication to a handheld communication device.
